(19) United States
(12) Patent Application Publication
(10) Pub. No.: US 2012/0115892 A1
(43) Pub. Date: May 10, 2012

(54) ADMINISTRATION OF NEDD8-ACTIVATING ENZYME INHIBITOR

(75) Inventors: Bruce J. Dezube, Newton, MA (US); Lilli M. Petruzzelli, Lexington, MA (US)

(73) Assignee: Millennium Pharmaceuticals, Inc., Cambridge, MA (US)

(21) Appl. No.: 13/288,146

(22) Filed: Nov. 3, 2011

Related U.S. Application Data
(60) Provisional application No. 61/410,478, filed on Nov. 5, 2010.
(60) Provisional application No. 61/488,240, filed on May 20, 2011.

(51) Int. Cl. A61K 31/519; A61P 35/00

(52) U.S. Cl. 514/265.1

(57) ABSTRACT

Disclosed are methods for the treatment of patients with diseases, disorders or conditions responsive to the inhibition of NAE (Nedd8-activating enzyme), e.g., cancer. The methods comprise administering a clinically effective amount of the NAE-inhibitor ((1S,2S,4R)-4-(4-((1S)-2,3-dihydro-1H-inden-1-ylamino)-7H-pyrrolo[2,3-d]pyrimidin-7-yl)-2-hydroxycyclopentyl)methyl sulfamate (MLN4924) according to an intermittent dosing regimen in which there is a rest period of at least two days between each administration.

This application claims the benefit under 35 U.S.C. §119(e) of U.S. Provisional Patent Application No. 61/410,478, filed Nov. 5, 2010 and U.S. Provisional Patent Application No. 61/488,240, filed May 20, 2011, both hereby incorporated by reference in their entirety.

### FIELD

The present invention relates to the field of oncology and provides methods for treating cancer.

### BACKGROUND

The compound ((1S,2S,4R)-4-(4-((1S)-2,3-dihydro-1H-inden-1-ylamino)-7H-pyrrolo[2,3-d]-pyrimidin-7-yl)-2-hydroxycyclopentyl)methyl sulfamate:

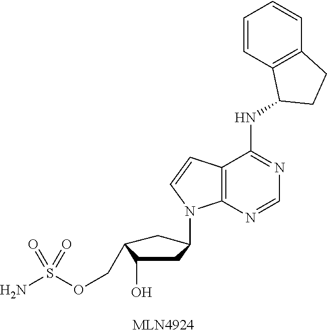

also known as MLN4924, is an inhibitor of NEDD8-activating enzyme (NAE). Inhibition of NAE has been shown to induce cancer cell death and inhibit the growth of tumors in xenograft models. See, e.g., T. A. Soucy et al., Nature, 2009, 458, 732-737; T. A. Soucy et al., Clin. Cancer Res., 2009, 15 (12), 3912-3916; and J. E. Brownell et al., Mol. Cell., 2010, 37 (1), 102-111, each of which is hereby incorporated by reference herein in its entirety. MLN4924, pharmaceutical compositions of MLN4924, processes for its synthesis, and polymorphic forms have been described previously. See, e.g., U.S. patent application Ser. Nos. 11/700,614 (Publ. No. 2007/0191293), 12/221,399 (Publ. No. 2009/0036678) and 12/779,331 (Publ. No. 2011/0021544), each of which is hereby incorporated by reference herein in its entirety. If there is any discrepancy between any of these documents and the present specification, the present specification controls.

As of Oct. 7, 2011, 193 patients have been treated in four ongoing MLN4924 Phase 1 clinical studies testing multiple different dosing schedules in both hematologic and nonhematologic tumors. Table 1 shows a summary of MTD (maximum tolerated dose) achieved with three of these dosing schedules.

TABLE 1

| Study | | | Dosing | | |
|---|---|---|---|---|---|
| C_{—} | Indication | n¹ | Schedule² | Dose (mg/m²)^{3,4} | |
| 15001 | solid | 12 | A: 1, 2, 3, 4, 5 | MTD | 50 |
| | tumors | 17 | B: 1, 3, 5 | MTD | 50 |
| | | | w/ dexa- | | |
| | | | methasone | | |
| | | 19 | C: 1, 3, 5 | MTD | 67 |
| | | | w/o dexa- | | |
| | | | methasone | | |
| 15002 | lymphoma, | 27 | A: 1, 2, 8, 9 | MTD | 110 |
| | MM⁵ | | | | |
| 15003 | AML⁵, MDS⁵ | 27 | A: 1, 3, 5 | MTD | 59 |

¹Number of patients enrolled
²Days of a 21-day schedule on which patient is dosed
³Amount of MLN4924 per patient BSA (body surface area)
⁴MTD noted if reached; “→” indicates MTD not reached
⁵MM = multiple myeloma; AML = acute myelogenous leukemia; MDS = myelodysplastic syndrome

Study C15001 is a first-in-human, phase 1 study in adults with advanced nonhematologic malignancies testing the tolerability of four 21-day dose schedules, including the following: Schedule A: QD×5; Schedule B: Days 1, 3, and 5 given with dexamethasone; and Schedule C: Days 1, 3, and 5 given without dexamethasone. Dose escalation is based on an adaptive approach using the continual reassessment method (CRM).

Schedule A (QD×5) testing started with 25 mg/m² and proceeded through 83 mg/m². Increases in liver function tests (LFTs) (AST, ALT, ALP and bilirubin) occurred during Cycle 1 following both single and multiple MLN4924 doses. LFT increases were dose limiting at doses exceeding 50 mg/m². Grade 3 and 4 LFT elevations occurred in 2 patients, who both died during the first week of study treatment. One death occurred in a patient with widely metastatic breast cancer receiving 61 mg/m², and the second occurred in a patient with advanced colon cancer receiving 83 mg/m². In both cases, the investigators assessed the hepatic toxicity as possibly related to MLN4924. As a result of these deaths and their association with increases in transaminase and bilirubin, all ongoing MLN4924 studies were amended to require that bilirubin levels be normal (<ULN) and that ALT and AST levels correspond with ≦Grade 1 intensity before administering initial and all subsequent doses of MLN4924. LFTs are checked prior to administration of every dose.

The MTD for Study C15001 Schedule A was determined to be 50 mg/m² QD×5. Based on observations in the ongoing C15002 study (see below) suggesting that a dosing regimen with rest periods between administrations may be better tolerated, C15001 was amended to add testing of dosing on Days 1, 3, and 5 repeated every 21 days. Also, based on study observations suggesting the presence of an acute phase reaction including transient, non-neutropenic fevers and increases in C-reactive protein (CRP), dexamethasone is co-administered with MLN4924 in C15001 Schedule B. C15001 Schedule C tests the same Days 1, 3, and 5 dose schedule but without dexamethasone. Three DLTs have occurred on Schedule B (Grade 3 LFTs), and 2 DLTs have occurred on Schedule C (Grade 3 LFTs). The MTD for these dosing schedules was determined to be 50 mg/m² and 67 mg/m², respectively.

Study C15002 is a phase 1 study in adults with lymphoma or multiple myeloma testing 3 dose schedules, each repeated on a 21-day cycle. One such schedule is Schedule A: Days 1, 2, 8, and 9. The MTD for Schedule A was determined to be 110 mg/m² based on DLTs consisting of febrile neutropenia occurring at 65 mg/m² and muscle cramps (Grade 4) and myalgia (Grade 2 but intolerable) occurring at 147 mg/m². One patient in the 110 mg/m² expansion cohort experienced Grade 3 AST and ALT elevation, but this did not contribute to the definition of MTD.

Study C15003 is a phase 1 study in adults with acute myelogenous leukemia and myelodysplastic syndrome testing 3 dose schedules, each repeated on a 21-day cycle. One such schedule is Schedule A: Days 1, 3 and 5. Dose escalation in C15003 is based on the standard 3+3 escalation approach. Five dose levels have been evaluated in Schedule A: 25 mg/m², 33 mg/m², 44 mg/m², 59 mg/m² and 78 mg/m². Two DLTs occurred at 78 mg/m² (multi-organ failure and reversible elevation of ALT). The MTD for Schedule A was determined to be 59 mg/m².

It is believed that efficacy of MLN4924 correlates with drug exposure, calculated as AUC. Accordingly, it is desirable to be able to administer MLN4924 at the highest dose possible, i.e., the highest dose at which the side-effect profile is acceptable. Therefore, a dosing regimen with a higher MTD than previously achieved would be expected to be able to produce a higher AUC, and thereby would be expected to provide a meaningful benefit in the treatment of patients with MLN4924.

### SUMMARY

It has now been discovered that MLN4924 can be administered so as generally to achieve a higher maximum tolerated dose, and thus at a higher clinically effective amount, than previously believed if it is administered using an intermittent dosing regimen, specifically, a dosing regimen in which there is a rest period of at least two days between each administration.

In one aspect, the present invention relates to a method of treating cancer in a patient, comprising administering to the patient a clinically effective amount of MLN4924 according to an intermittent dosing regimen, wherein (a) the intermittent dosing regimen comprises a rest period of at least two days between each administration; and (b) the clinically effective amount is (i) greater than or equal to about 145 mg/m² and (ii) less than or equal to the maximum tolerated dose (MTD).

In one aspect, the present invention relates to MLN4924 for use in a method of treating cancer in a patient wherein a clinically effective amount of MLN4924 is administered to the patient according to an intermittent dosing regimen, wherein: (a) the intermittent dosing regimen comprises a rest period of at least two days between each administration; and (b) the clinically effective amount is (i) greater than or equal to about 145 mg/m² and (ii) less than or equal to the maximum tolerated dose (MTD).

### DESCRIPTION

### Definitions and Abbreviations

ALP alkaline phosphatase
ALT alanine aminotransferase
AML acute myelogenous leukemia
ANC absolute neutrophil count
AST aspartate aminotransferase
AUC area under the plasma concentration versus time curve
BSA body surface area
CR complete response
CRM continual reassessment method
CYP cytochrome P450
DLBCL diffuse large B-cell lymphoma
DLT dose-limiting toxicity
LFT liver function tests
LVEF left ventricular ejection fraction
MDS myelodysplastic syndrome
MM multiple myeloma
MTD maximum tolerated dose
NAE Nedd8-activating enzyme
Nedd8 neural precursor cell expressed, developmentally down-regulated 8
PASP pulmonary artery systolic pressure
PR partial response
QD once daily
SCLC small cell lung cancer

As used herein, “body surface area” (BSA) is calculated using a standard nomogram, e.g.,

$$B S A ( {m}^{2} ) = \sqrt{\frac{Ht ( cm ) \times Wt ( kg )}{3600}} or B S A = \sqrt{\frac{Ht ( in ) \times Wt ( lb )}{3131}}$$

In any form or composition, the clinically effective amount, the MTD and the administered dose can each be expressed as amount of therapeutic substance per patient BSA, e.g., as mg/m².

As used herein, “dose-limiting toxicity” (DLT) is defined as any of the following events that are considered by the administering physician to be related to therapy with MLN4924:
Grade 4 neutropenia (ANC<500 cells/mm³) lasting more than 7 consecutive days
Grade 3 neutropenia with fever and/or infection, where fever is defined as an oral temperature ≧38.5° C.
Grade 4 thrombocytopenia (platelets <25,000/mm³ but >10,000/mm³) lasting more than 7 consecutive days
Grade 3 thrombocytopenia with bleeding
A platelet count <10,000/mm³ at any time
Grade 3 or greater nausea and/or emesis despite the use of optimal anti-emetic prophylaxis (wherein “optimal anti-emetic prophylaxis” is defined as an anti-emetic regimen that employs a 5-HT₃ antagonist given in standard doses and according to standard schedules). Dexamethasone should not be used because of its CYP3A-inducing effects.
Grade 3 or greater diarrhea that occurs despite maximal supportive therapy
An absolute reduction in LVEF of ≧10% to a value <50% (e.g., LVEF=45% in a patient with LVEF=55% at baseline)
A decrease in LVEF to <40%
An increase in PASP to >50 mm Hg or 3× baseline
Any other Grade 3 or greater nonhematologic toxicity with the following exceptions:
   Grade 3 arthralgia/myalgia
   Brief (<1 week) Grade 3 fatigue
   Grade 3 fever that occurs in the absence of Grade 3 or worse neutropenia or documented infection following daily administration of MLN4924
Treatment delay of more than 1 week because of a lack of adequate recovery of MLN4924-related hematological or nonhematologic toxicities
MLN4924-related toxicity that requires that any doses of MLN4924 are missed during a cycle or discontinuation of therapy with MLN4924

As used herein, “clinically effective amount” means an amount of a therapeutic substance (e.g., MLN4924) that is (1) sufficient upon appropriate administration to a patient (a) to cause a detectable decrease in the severity of the disorder or disease state being treated; (b) to ameliorate or alleviate the patient's symptoms of the disease or disorder; or (c) to slow or prevent advancement of, or otherwise stabilize or prolong stabilization of, the disorder or disease state being treated (e.g., prevent additional tumor growth of a cancer); and (2) equal to or less than the MTD.

As used herein, “patient” means a human being diagnosed with, exhibiting symptoms of or otherwise believed to be afflicted with a disease, disorder or condition.

As used herein, the term “about”, when preceding a series of numbers, is intended to modify each of the numbers in the series. For example, “about 10, 20 or 30” means the same as “about 10, about 20 or about 30”.

As used herein, the illustrative terms “include”, “such as”, “for example” and the like (and variations thereof, e.g., “includes” and “including”, “examples”), unless otherwise specified, are intended to be non-limiting. That is, unless explicitly stated otherwise, such terms are intended to imply “but not limited to”, e.g., “including” means including but not limited to.

### Dosing Regimens.

It has now been discovered that MLN4924 can be administered so as generally to achieve a higher maximum tolerated dose, and thus at a higher clinically effective amount, than previously believed if it is administered using an intermittent dosing regimen, specifically, a dosing regimen in which there is a rest period of at least two days between each administration. Table 2 shows a summary of MTD (maximum tolerated dose) with different dosing schedules, including such intermittent dosing schedules.

TABLE 2

| Study | | | Dosing | | |
|---|---|---|---|---|---|
| C_{—} | Indication | n¹ | Schedule² | Dose (mg/m²)^{3,4} | |
| 15001 | solid | 12 | A: 1, 2, 3, 4, 5 | MTD | 50 |
| | tumors | 17 | B: 1, 3, 5 | MTD | 50 |
| | | | w/ dexa- | | |
| | | | methasone | | |
| | | 19 | C: 1, 3, 5 | MTD | 67 |
| | | | w/o dexa- | | |
| | | | methasone | | |
| | | 6 | D: 1, 8, 15 | → | 196 |
| | | | 3-hr infusn. | | |
| 15002 | lymphoma, | 27 | A: 1, 2, 8, 9 | MTD | 110 |
| | MM⁵ | 16 | B: 1, 4, 8, 11 | MTD | 196 |
| | | 9 | C: 1, 8 | * | * |
| 15003 | AML⁵, MDS⁵ | 27 | A: 1, 3, 5 | MTD | 59 |
| | | 22 | B: 1, 4, 8, 11 | MTD | 83 |
| | | 2 | C: 1, 8, 15 | * | * |
| 15005 | melanoma | 25 | A: 1, 4, 8, 11 | MTD | 209 |
| | | 11 | B: 1, 8, 15 | * | * |

¹Number of patients enrolled
²Days of a 21-day schedule on which patient is dosed
³Amount of MLN4924 per patient BSA (body surface area)
⁴MTD noted if reached; “→” indicates MTD not reached
⁵MM = multiple myeloma; AML = acute myelogenous leukemia; MDS = myelodysplastic syndrome;
*Study closed without determination

Study C15001 is a phase 1 study in adults with advanced nonhematologic malignancies testing the tolerability of four 21-day dose schedules, including one intermittent dosing schedule, Schedule D, on which doses are administered on Days 1, 8, and 15 as a 3-hour infusion. As of Oct. 7, 2011, one DLT has been observed. Patients have been enrolled to the following dose levels: 147 mg/m² and 196 mg/m². Testing continues, and dose escalation is based on an adaptive approach using the continual reassessment method (CRM).

Study C15002 is a phase 1 study in adults with lymphoma or multiple myeloma testing 3 dose schedules, each repeated on a 21-day cycle. Two such schedules are intermittent dosing schedules, Schedule B: Days 1, 4, 8, and 11; and Schedule C: Days 1 and 8. As of Oct. 7, 2011, no DLTs have been observed on Schedule B. One DLT occurred on Schedule C at 196 mg/m² (reversible Grade 3 renal failure), which has since been closed. Patients on Schedule B were enrolled to dose levels of 110 mg/m², 147 mg/m², 196 mg/m² and 261 mg/m² before the MTD was determined to be 196 mg/m².

Study C15005 is a phase 1, dose-escalation study in adults with metastatic melanoma testing MLN4924 dosing on Days 1, 4, 8, and 11 (Schedule A) or Days 1, 8 and 15 (Schedule B) repeated every 21 days. As of Oct. 7, 2011, two DLTs have occurred on Schedule A. One patient at the 118 mg/m² dose level experienced MLN4924-related grade 3 hypophosphatemia, resulting in a dose reduction of MLN4924. One patient at the 278 mg/m² dose level experienced a completely reversible, grade 3 increase in creatinine and bilirubin after day 1 of cycle 1. Patients were enrolled to dose levels of 50 mg/m², 67 mg/m², 89 mg/m², 118 mg/m², 157 mg/m², 209 mg/m² and 278 mg/m² before the MTD was determined to be 209 mg/m². Prior to discontinuation of Schedule B, two DLTs, including myocarditis and increased blood creatinine, were observed at 157 mg/m².

Study C15003 is a phase 1 study in adults with acute myelogenous leukemia and myelodysplastic syndrome testing 3 dose schedules, each repeated on a 21-day cycle. One such schedule is an intermittent dosing schedule, Schedule B: Days 1, 4, 8, and 11, for which the MTD was determined to be 83 mg/m². Four DLTs in three patients have occurred at the 147 mg/m² dose level. They include lactic acidosis and hypotension, GI necrosis, and acute renal failure. A second intermittent dosing schedule, Schedule C, which was to evaluate continuous weekly dosing on Days 1, 8 and 15 was closed without determination of MTD.

Thus, in one aspect, the present invention relates to a method of treating cancer in a patient, comprising administering to the patient a clinically effective amount of MLN4924 according to an intermittent dosing regimen, wherein (a) the intermittent dosing regimen comprises a rest period of at least two days between each administration; and (b) the clinically effective amount is (i) greater than or equal to about 145 mg/m² and (ii) less than or equal to the maximum tolerated dose (MTD).

In one aspect, the present invention relates to MLN4924 for use in a method of treating cancer in a patient wherein a clinically effective amount of MLN4924 is administered to the patient according to an intermittent dosing regimen, wherein: (a) the intermittent dosing regimen comprises a rest period of at least two days between each administration; and (b) the clinically effective amount is (i) greater than or equal to about 145 mg/m² and (ii) less than or equal to the maximum tolerated dose (MTD).

In some embodiments, the invention comprises administering MLN4924 according to the intermittent dosing regimen on each of days 1, 4, 8 and 11 of a 21-day cycle. In some embodiments, the invention comprises administering MLN4924 according to the intermittent dosing regimen on each of days 1, 8 and 15 of a 21-day cycle. In some embodiments, the invention comprises administering MLN4924 according to the intermittent dosing regimen on each of days 1 and 8 of a 21-day cycle.

In various embodiments, the clinically effective amount is greater than or equal to about: 145 mg/m², 155 mg/m², 165 mg/m², 175 mg/m², 185 mg/m², 190 mg/m², 200 mg/m², 210 mg/m², 220 mg/m² or 230 mg/m². In various embodiments, the MTD is about: 275 mg/m², 270 mg/m², 265 mg/m², 260 mg/m², 255 mg/m², 250 mg/m², 245 mg/m², 240 mg/m², 230 mg/m², 220 mg/m², 209 mg/m² or 196 mg/m².

The MTD can be determined by one skilled in the art, and can vary depending on the indication. For example, it has been determined that the MTD for treating melanoma using a 1-hr intravenous infusion on each of days 1, 4, 8 and 11 of a 21-day cycle is about 209 mg/m². Accordingly, in various embodiments of treating melanoma by administering MLN4924 using a 1-hr intravenous infusion on each of days 1, 4, 8 and 11 of a 21-day cycle, the clinically effective amount is less than or equal to about 209 mg/m², and greater than or equal to about: 145 mg/m², 155 mg/m², 165 mg/m², 175 mg/m², 185 mg/m², 190 mg/m² or 200 mg/m².

Similarly, it has been determined that the MTD for treating lymphoma and/or multiple myeloma using a 1-hr intravenous infusion on each of days 1, 4, 8 and 11 of a 21-day cycle is about 196 mg/m². Accordingly, in various embodiments of treating lymphoma and/or multiple myeloma by administering MLN4924 using a 1-hr intravenous infusion on each of days 1, 4, 8 and 11 of a 21-day cycle, the clinically effective amount is less than or equal to about 196 mg/m², and greater than or equal to about: 145 mg/m², 155 mg/m², 165 mg/m², 175 mg/m², 185 mg/m² or 190 mg/m².

In some embodiments, the invention comprises administering MLN4924 orally according to the intermittent dosing regimen, and additionally according to any of the foregoing 21-day administration cycles. In some embodiments, the invention comprises administering MLN4924 intravenously according to the intermittent dosing regimen, and additionally according to any of the foregoing 21-day administration cycles. In various embodiments, the intravenous administration comprises an infusion of about: 60 minutes, 90 minutes, 2 hours, 2.5 hours, 3 hours, 3.5 hours, 4 hours, 5 hours or 6 hours.

In various embodiments, the invention comprises treating cancer, wherein the cancer is a solid tumor, by administering MLN4924 using a 3-hr intravenous infusion, wherein the clinically effective amount is less than or equal to about 196 mg/m², and greater than or equal to about: 145 mg/m², 155 mg/m², 165 mg/m², 175 mg/m², 185 mg/m² or 190 mg/m²; less than or equal to about 261 mg/m², and greater than or equal to about: 145 mg/m², 155 mg/m², 165 mg/m², 175 mg/m², 185 mg/m², 190 mg/m², 200 mg/m², 210 mg/m², 220 mg/m² or 230 mg/m²; or less than or equal to the MTD, and greater than or equal to about: 145 mg/m², 155 mg/m², 165 mg/m², 175 mg/m², 185 mg/m², 190 mg/m², 200 mg/m², 210 mg/m², 220 mg/m² or 230 mg/m². In some such embodiments, the invention comprises administering MLN4924 using a 3-hr infusion on each of days 1, 8 and 15 of a 21-day cycle. In some such embodiments, the solid tumor is small-cell lung cancer, colon cancer or melanoma.

### Therapeutic Substance; Pharmaceutical Compositions.

The therapeutic substance can be a pharmaceutically acceptable salt. In some embodiments, such salts are derived from inorganic or organic acids or bases. For reviews of suitable salts, see, e.g., Berge et al., J. Pharm. Sci., 1977, 66, 1-19 and Remington: The Science and Practice of Pharmacy, 20th Ed., A. Gennaro (ed.), Lippincott Williams & Wilkins (2000).

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydro-abietyl)ethylenediamine), hydrobromide, hydrochloride, hydroxynaphthoate, iodide, isethionate (2-hydroxyethanesulfonate), lactate, lactobionate, malate, maleate, mandelate, mesylate (methane-sulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolidine-1′-ylmethylbenzimidazole), diethylamine, piperazine and tromethamine (tris(hydroxymethyl)aminomethane); and metallic cations barium and bismuth.

As used herein, “pharmaceutically acceptable carrier” refers to a material that is compatible with a recipient subject (a human) and is suitable for delivering an active agent to the target site without terminating the activity of the agent. The toxicity or adverse effects, if any, associated with the carrier preferably are commensurate with a reasonable risk/benefit ratio for the intended use of the active agent.

The pharmaceutical compositions for use in the invention can be manufactured by methods well known in the art such as conventional granulating, mixing, dissolving, encapsulating, lyophilizing, or emulsifying processes, among others. Compositions can be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations can contain stabilizers, pH modifiers, surfactants, solubilizing agents, bioavailability modifiers and combinations of these.

Pharmaceutically acceptable carriers that can be used in these compositions include ion exchangers, alumina, aluminum stearate, lecithin, serum proteins, such as human serum albumin, buffer substances such as phosphates or carbonates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

These pharmaceutical compositions are formulated for pharmaceutical administration to a human being. Such compositions can be administered orally, parenterally, by inhalation spray, topically, rectally, nasally, buccally, vaginally or via an implanted reservoir. The term “parenteral” as used herein includes subcutaneous, intravenous, intra peritoneal, intramuscular, intra-articular, intra-synovial, intrasternal, intrathecal, intrahepatic, intralesional and intracranial injection or infusion techniques. In some embodiments, the compositions are administered orally, intravenously or subcutaneously. In some embodiments, the compositions are administered orally. In some embodiments, the compositions are administered intravenously. These formulations can be designed to be short-acting, fast-releasing, or long-acting. Furthermore, the compositions can be administered in a local rather than systemic means, such as administration (e.g., by injection) at a tumor site.

Pharmaceutical formulations can be prepared as liquid suspensions or solutions using a liquid, such as an oil, water, an alcohol, and combinations of these. Solubilizing agents such as cyclodextrins can be included. Pharmaceutically suitable surfactants, suspending agents, or emulsifying agents, can be added for oral or parenteral administration. Suspensions can include oils, such as peanut oil, sesame oil, cottonseed oil, corn oil and olive oil. Suspension preparations can also contain esters of fatty acids such as ethyl oleate, isopropyl myristate, fatty acid glycerides and acetylated fatty acid glycerides. Suspension formulations can include alcohols, such as ethanol, isopropyl alcohol, hexadecyl alcohol, glycerol and propylene glycol; ethers, such as poly(ethyleneglycol); petroleum hydrocarbons such as mineral oil and petrolatum; and water.

Sterile injectable forms of these pharmaceutical compositions can be aqueous or oleaginous suspensions. These suspensions may be formulated according to techniques known in the art using suitable dispersing or wetting agents and suspending agents. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation. Compounds can be formulated for parenteral administration by injection such as by bolus injection or continuous infusion. A unit dosage form for injection can be in ampoules or in multi-dose containers.

These pharmaceutical compositions can be orally administered in any orally acceptable dosage form including capsules, tablets, aqueous suspensions or solutions. When aqueous suspensions are required for oral use, the active ingredient is combined with emulsifying and suspending agents. If desired, certain sweetening, flavoring or coloring agents can also be added. For oral administration in a capsule form, useful diluents include lactose and dried cornstarch. In the case of tablets for oral use, carriers that are commonly used include lactose and corn starch. Lubricating agents, such as magnesium stearate, are also typically added. Coatings may be used for a variety of purposes, e.g., to mask taste, to affect the site of dissolution or absorption, or to prolong drug action. Coatings can be applied to a tablet or to granulated particles for use in a capsule.

Alternatively, these pharmaceutical compositions can be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

These pharmaceutical compositions can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches can also be used. For topical applications, the pharmaceutical compositions can be formulated in a suitable ointment containing the active component suspended or dissolved in one or more carriers. Carriers for topical administration of the compounds of this invention include mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active component(s) suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, the pharmaceutical compositions can be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions can be formulated in an ointment such as petrolatum.

The pharmaceutical compositions can also be administered by nasal aerosol or inhalation. Such compositions are prepared according to techniques well known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

The methods of the invention are directed to treating diseases, disorders and conditions in which inhibition of NAE enzyme activity is detrimental to survival and/or expansion of diseased cells or tissue (e.g., cells are sensitive to NAE inhibition; inhibition of NAE activity disrupts disease mechanisms; reduction of NAE activity stabilizes protein which are inhibitors of disease mechanisms; reduction of NAE activity results in inhibition of proteins which are activators of disease mechanisms). The diseases, disorders and conditions are also intended to include those which require effective cullin and/or ubiquitination activity, which activity can be regulated by diminishing NAE enzyme activity.

For example, methods of the invention are useful in treatment of disorders involving cellular proliferation, including disorders which require an effective cullin-dependent ubiquitination and proteolysis pathway (e.g., the ubiquitin proteasome pathway) for maintenance and/or progression of the disease state. The methods of the invention are useful in treatment of disorders mediated via proteins (e.g., NFκB activation, p27^{Kip} activation, p21^{WAF/CIP1} activation, p53 activation) which are regulated by NAE activity. Relevant disorders include proliferative disorders, most notably cancers and inflammatory disorders (e.g., rheumatoid arthritis, inflammatory bowel disease, asthma, chronic obstructive pulmonary disease (COPD), osteoarthritis, dermatosis (e.g., atopic dermatitis, psoriasis), vascular proliferative disorders (e.g., atherosclerosis, restenosis) autoimmune diseases (e.g., multiple sclerosis, tissue and organ rejection)); as well as inflammation associated with infection (e.g., immune responses), neurodegenerative disorders (e.g., Alzheimer's disease, Parkinson's disease, motor neuron disease, neuropathic pain, triplet repeat disorders, astrocytoma, and neurodegeneration as result of alcoholic liver disease), ischemic injury (e.g., stroke), and cachexia (e.g., accelerated muscle protein breakdown that accompanies various physiological and pathological states, (e.g., nerve injury, fasting, fever, acidosis, HIV infection, cancer affliction, and certain endocrinopathies)).

The methods of the invention are particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes solid tumors and bloodborne tumors. The term “cancer” encompasses diseases of skin, tissues, organs, bone, cartilage, blood, and vessels. The term “cancer” further encompasses primary and metastatic cancers.

In some embodiments, the cancer is a solid tumor. Examples of solid tumors that can be treated by the methods of the invention include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., small cell lung cancer (SCLC), non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

In some embodiments, the cancer is a hematologic malignancy. Examples of hematologic malignancy include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In some embodiments, methods of the invention are used to treat a patient having or at risk of developing or experiencing a recurrence in a cancer selected from colorectal cancer, ovarian cancer, lung cancer, breast cancer, gastric cancer, prostate cancer and pancreatic cancer. In some embodiments, the cancer is selected from lung cancer, colorectal cancer, ovarian cancer and a hematologic cancer.

In some embodiments, depending on the particular disorder or condition to be treated, MLN4924 is administered in conjunction with an additional therapeutic agent or agents. In some embodiments, the additional therapeutic agent(s) is one that is normally administered to patients with the disorder or condition being treated. As used herein, additional therapeutic agents that are normally administered to treat a particular disorder or condition are known as “appropriate for the disorder or condition being treated.”

MLN4924 can be administered with the other therapeutic agent in a single dosage form or as a separate dosage form. When administered as a separate dosage form, the other therapeutic agent can be administered prior to, at the same time as, or following administration of MLN4924.

In some embodiments, MLN4924 is administered in conjunction with a therapeutic agent selected from cytotoxic agents, radiotherapy, and immunotherapy appropriate for treatment of proliferative disorders and cancer. Examples of cytotoxic agents suitable for use in combination with MLN4924 include: antimetabolites, including, e.g., capecitibine, gemcitabine, 5-fluorouracil or 5-fluorouracil/leucovorin, fludarabine, cytarabine, mercaptopurine, thioguanine, pentostatin, and methotrexate; topoisomerase inhibitors, including, e.g., etoposide, teniposide, camptothecin, topotecan, irinotecan, doxorubicin, and daunorubicin; vinca alkaloids, including, e.g., vincristine and vinblastin; taxanes, including, e.g., paclitaxel and docetaxel; platinum agents, including, e.g., cisplatin, carboplatin, and oxaliplatin; antibiotics, including, e.g., actinomycin D, bleomycin, mitomycin C, adriamycin, daunorubicin, idarubicin, doxorubicin and pegylated liposomal doxorubicin; alkylating agents such as melphalan, chlorambucil, busulfan, thiotepa, ifosfamide, carmustine, lomustine, semustine, streptozocin, decarbazine, and cyclophosphamide; including, e.g., CC-5013 and CC-4047; protein tyrosine kinase inhibitors, including, e.g., imatinib mesylate and gefitinib; proteasome inhibitors, including, e.g., bortezomib, thalidomide and related analogs; antibodies, including, e.g., trastuzumab, rituximab, cetuximab, and bevacizumab; mitoxantrone; dexamethasone; prednisone; and temozolomide.

Other examples of agents with which MLN4924 can be combined with include anti-inflammatory agents such as corticosteroids, TNF blockers, Il-RA, azathioprine, cyclophosphamide, and sulfasalazine; immunomodulatory and immunosuppressive agents such as cyclosporine, tacrolimus, rapamycin, mycophenolate mofetil, interferons, corticosteroids, cyclophosphamide, azathioprine, methotrexate, and sulfasalazine; antibacterial and antiviral agents; and agents for Alzheimer's treatment such as donepezil, galantamine, memantine and rivastigmine.

In order that this invention be more fully understood, the following examples are set forth. These examples are illustrative only and are not intended to limit the scope of the invention in any way.

### EXAMPLES

### Dosage Form.

MLN4924 Drug Substance (“MLN4924-DS”) is the hydrochloride salt of MLN4924, i.e., ((1S,2S,4R)-4-(4-((1S)-2,3-dihydro-1H-inden-1-ylamino)-7H-pyrrolo[2,3-d]pyrimidin-7-yl)-2-hydroxy-cyclopentyl)methyl sulfamate hydrochloride.

MLN4924 Injection Drug Product (“MLN4924-IDP”) is formulated with the following excipients: citric acid; sodium hydroxide; Cyclodextrin Sulfobutylethers, Sodium Salts (Captisol®); and water for injection. The formulation consists of 10 mg/mL MLN4924 (as free base) in a solution containing 50 mM citrate buffer and 100 mg/mL sulfobutylether β-cyclodextrin, pH 3.3.

MLN4924-IDP is not stable when diluted in saline. MLN4924-IDP can be used for the duration of the retest period indicated on the Certificate of Analysis. MLN4924-IDP must be stored refrigerated at 5° C.±3° C.

Each Type I glass vial nominally contains 5 mL of compounded sterile solution, sealed with a Teflon®-coated butyl rubber stopper and oversealed with an aluminum seal with a plastic Flip-Off® cap.

### Instructions for Use; Drug Administration.

Prior to use, MLN4924-IDP vials are warmed to ambient conditions (15° C. to 30° C.) by placing them at room temperature. Accelerated warming methods such as a water bath must not be used. MLN4924-IDP is stable at room temperature for 8 hours prior to dilution.

Each MLN4924-IDP vial contains nominally 5 mL (50 mg MLN4924 as free base). Using sterile technique, the appropriate volume of drug is withdrawn from vial(s) and injected into a 250 mL IV bag containing a 5% dextrose solution, which is then gently inverted repeatedly to mix. The prepared MLN4924-IDP IV bag must be used within 6 hours if stored at room temperature. Alternatively, the prepared IV bag is chemically stable and can be stored for up to 24 hours at 5° C.±3° C. After 24 hours of storage at 5° C.±3° C., the prepared IV bag must be used within 6 hours upon coming to room temperature. The vial must not be shaken at any time during dose preparation.

The amount of MLN4924 administered is based on body surface area. BSA is calculated using a standard nomogram on Cycle 1, Day 1, and at subsequent visits if the patient experiences a >5% change in body weight from the weight used for the most recent BSA calculation.

Patients receive MLN4924 diluted with 5% dextrose in a 250-mL IV bag via a 60-minute infusion on Days 1, 4, 8 and 11 or Days 1, 8 and 15 of a 21-day cycle, or via a 3-hour infusion on Days 1, 8 and 15 of a 21-day cycle. Doses of MLN4924 must be separated by at least two days. MLN4924 should be administered through central or peripheral venous access. The infusion can be slowed or stopped and restarted for any associated infusion reactions. The total infusion time must not exceed six hours from the time of reconstitution.

Although DLTs can occur at any point during treatment, only DLTs occurring during Cycle 1 of treatment will necessarily influence decisions regarding dose escalation, expansion of a dose level, or evaluation of intermediate dose levels. Patients are monitored through all cycles of therapy for treatment-related toxicities.

Table 3 summarizes the positive findings for each of the clinical studies as of Oct. 16, 2011.

TABLE 3

| Study | | | Dosing | | | |
|---|---|---|---|---|---|---|
| C | Indication | n¹ | Schedule² | Dose (mg/m²)^{3,4} | | Findings |
| 15001 | Solid | 12 | A: 1, 2, 3, 4, 5 | MTD | 50 | Prolonged stable disease in |
| | Tumors | 17 | B: 1, 3, 5 | MTD | 50 | SCLC⁵ (7 cycles), |
| | | | w/ dexa- | | | CRC⁵ (5, 5, 9 cycles), |
| | | | methasone | | | Breast (5, 7 cycles) |
| | | 19 | C: 1, 3, 5 | MTD | 67 | Head and Neck (7 cycles) |
| | | | w/o dexa- | | | Melanoma (7, 7, 10 cycles) |
| | | | methasone | | | |
| | | 6 | D: 1, 8, 15 | → | 196 | Prolonged stable disease in |
| | | | 3-hr infusn. | | | Colon cancer (6+ cycles) |
| | | | | | | NSCLC⁵ (5+ cycles) |
| 15002 | Lymphoma, | 27 | A: 1, 2, 8, 9 | MTD | 110 | Partial remission in Hodgkin's |
| | MM⁵ | 16 | B: 1, 4, 8, 11 | MTD | 196 | Lymphoma (7 cycles); |
| | | | | | | Partial remission in DLBCL⁵ and |
| | | | | | | PTCL⁵ |
| 15003 | AML⁵, MDS⁵ | 27 | A: 1, 3, 5 | MTD | 59 | 3 CRs⁶ during dose escalation; |
| | | | | | | one patient received 17 cycles |
| | | 22 | B: 1, 4, 8, 11 | MTD | 83 | |
| 15005 | Melanoma | 25 | A: 1, 4, 8, 11 | MTD | 209 | Prolonged stable disease in |
| | | | | | | Melanoma (5, 6, 6, 6, 10, 12 cycles) |
| | | 11 | B: 1, 8, 15 | * | * | Prolonged stable disease in |
| | | | | | | Melanoma (11+ cycles) |

¹Number of patients enrolled
²Days of a 21-day schedule on which patient is dosed
³Amount of MLN4924 per patient BSA (body surface area)
⁴MTD noted if reached; “→” indicates MTD not reached
⁵SCLC = small cell lung cancer; MM = multiple myeloma; DLBCL = diffuse large B-cell lymphoma; AML = acute myelogenous leukemia; MDS = myelodysplastic syndrome; CRC = colorectal cancer; NSCLC = non-small cell lung cancer; PTCL = peripheral T-cell lymphoma
⁶CR = complete response
* Study closed without determination

What is claimed is:

1. A method of treating cancer in a patient in need of such treatment, comprising administering to the patient a clinically effective amount of MLN4924 according to an intermittent dosing regimen, wherein:
(a) the intermittent dosing regimen comprises a rest period of at least two days between each administration; and
(b) the clinically effective amount is (i) greater than or equal to about 145 mg/m² and (ii) less than or equal to the maximum tolerated dose (MTD).

2. The method of claim 1, wherein the clinically effective amount is greater than or equal to about 175 mg/m².

3. The method of claim 1, wherein the clinically effective amount is greater than or equal to about 190 mg/m².

4. The method of claim 1, wherein the MTD is about 209 mg/m².

5. The method of claim 1, wherein the MTD is about 196 mg/m².

6. The method of claim 1, wherein the intermittent dosing regimen comprises administering MLN4924 on each of Days 1, 4, 8 and 11 of a 21-day cycle.

7. The method of claim 1, wherein the intermittent dosing regimen comprises administering MLN4924 on each of Days 1, 8 and 15 of a 21-day cycle.

8. The method of claim 1, wherein MLN4924 is administered intravenously.

9. The method of claim 8, wherein the intravenous administration comprises a 60-minute infusion.

10. The method of claim 8, wherein the intravenous administration comprises a 3-hour infusion.

11. The method of claim 1, wherein the cancer is small-cell lung cancer, colon cancer, melanoma, Hodgkin's lymphoma, diffuse large B-cell lymphoma, follicular lymphoma or multiple myeloma.

12. The method of claim 1, wherein the cancer is melanoma.

13. The method of claim 12, wherein the intermittent dosing regimen comprises administering MLN4924 on each of days 1, 4, 8 and 11 of a 21-day cycle.

14. The method of claim 13, wherein MLN4924 is administered intravenously.

15. The method of claim 14, wherein the intravenous administration comprises a 60-minute infusion.

16. The method of claim 15, wherein the MTD is about 209 mg/m².

17. The method claim 1, wherein the cancer is lymphoma or multiple myeloma.

18. The method of claim 17, wherein the intermittent dosing regimen comprises administering MLN4924 on each of days 1, 4, 8 and 11 of a 21-day cycle.

19. The method of claim 18, wherein MLN4924 is administered intravenously.

20. The method of claim 19, wherein the intravenous administration comprises a 60-minute infusion.

21. The method of claim 20, wherein the MTD is about 196 mg/m².

22. The method of claim 10, wherein the cancer is a solid tumor.

23. The method of claim 22, wherein the cancer is small-cell lung cancer, colon cancer or melanoma.

24. The method of claim 22, wherein the intermittent dosing regimen comprises administering MLN4924 on each of days 1, 8 and 15 of a 21-day cycle.
